(19) United States
(12) Patent Application Publication
(10) Pub. No.: US 2015/0202311 A1
(43) Pub. Date: Jul. 23, 2015

(54) Collagen-peptide complex

(71) Applicant: Q'SAI CO., LTD., Fukuoka (JP)

(72) Inventors: Asahi SUZUKI, Fukuoka (JP); Mihoko KUROKAWA, Fukuoka (JP); Yasutaka SHIGEMURA, Tokyo (JP)

(21) Appl. No.: 14/571,431

(22) Filed: Dec. 16, 2014

(30) Foreign Application Priority Data
Dec. 17, 2013 (JP) 2013-260232

(51) Int. Cl. A61K 47/48; A23L 1/305

(52) U.S. Cl. CPC A61K 47/48061; A23L 1/3053; A23V 2002/00

(57) ABSTRACT

[Theme] An object is to provide a collagen-containing composition for oral ingestion wherein the resulting peptide-type Hyp is effectively transferred into the blood.

[Means for solving the problem] A water-insoluble complex comprising a water-soluble collagen peptide complexed with theaflavins and/or epicatechin.

### TECHNICAL FIELD

The present invention relates to complexes of collagen peptide and catechins, and in particular, relates to collagen peptide/catechin complexes for oral ingestion wherein the resulting peptide-type collagen is effectively transferred into the blood.

### BACKGROUND ART

Collagen is a kind of protein that constitutes the dermis, ligament, tendon, bone, cartilage, etc., and collagen is the main component of the extracellular substrate (extracellular matrix) of multicellular organisms. A human has a plural number of collagen genes, and 30 or more collagen proteins have been identified. Among them, type 1 collagen is present in the largest amount in the body, in which three polypeptide chains consisting of about 1,000 amino acids (two α1 chains and one α2 chain) are helically associated and formed a fibrous structure called “collagen fibers. The molecular weight of the collagen fiber is about 300K (=about 100K×3), and it is nearly insoluble in water (non-patent literature 1).

The composition and sequence of amino acids in collagen have marked characteristics, the percentage of glycine (Gly) is about 30%, the combined percentage of proline (Pro) and hydroxyproline (Hyp) is about 21%, the percentage of alanine is about 11%, and “Gly-X-Y (X and Y are arbitrary amino acids, respectively)” is repeated about 330 times. Hydroxyproline (hereinafter, it may be abbreviated to “Hyp”) is an amino acid characteristic of collagen, and Hyp has been used as an indicator for the identification/quantification of collagen and collagen hydrolysate (namely, collagen peptide) since old days (non-patent literature 1).

Protein ingested as food is normally digested into polypeptides with pepsin in the stomach and further digested into free amino acids by the action of trypsin, chymotrypsin, elastase, carboxypeptidase, etc. in the duodenum and aminopeptidase, dipeptidase, etc. in the small intestine. The amino acids are absorbed into small-intestinal epithelial cells and then transferred into the blood through the amino acid transporters on the membrane of the epithelial cells. Furthermore, it is known that they can be transferred into the blood through the peptide transporters on the epithelial cell membrane even in the state of tripeptides and dipeptides.

As described above, collagen fibers are nearly insoluble in water and resistant to digestion. Therefore, gelatin obtained by partial hydrolysis of collagen fibers by heat denaturation and the low-molecular weight collagen peptides obtained by further enzymatic hydrolysis have been widely distributed in the food market. In particular, the effects such as the alleviation of joint pain, an increase in bone density, a reduction of skin damage by ultraviolet irradiation (decrease in the stratum corneum moisture content, epidermal hyperplasia, reduction of dermal collagen, etc.), due to oral ingestion of collagen peptides, have been reported (non-patent literature 1), and the expectation as health food is high.

When a human orally ingests collagen peptides, the most is absorbed as amino acids. However, Hyp-containing tripeptides and/or dipeptides (hereinafter, they may be called “peptide-type Hyp”) are detected in the blood 1-2 hours alter the ingestion; thus it is known that some is absorbed and transferred into the blood in the state of tri-/dipeptides (non-patent literature 2). In the recent studies, it was clarified that the peptide-type Hyp achieves beneficial physiological activity for various cells.

For example, it is reported that when Pro-Hyp dipeptide, which is a representative of the peptide-type Hyp in the blood, is administered to a piece of mouse skin section in culture, the migratory capacity and growth capacity of the skin fibroblasts are promoted (non-patent literature 2). Pro-Hyp also enhances the expression of hyaluronan synthase 2 and promotes cell proliferation in the human primary fibroblasts (non-patent literature 3). In addition, Pro-Hyp can inhibit a calcification of the mouse cartilage precursor cell line (non-patent literature 4) and can induce a differentiation of the mouse osteoblast-like cell line to osteoblasts (non-patent literature 5).

From these research results, many of the effects obtained by orally ingesting collagen or collagen peptides are considered to be mainly due to the peptide-type Hyp transferred into the blood.

However, when the peptide-type Hyp is orally ingested, it is usually completely hydrolyzed to amino acids; thus the absorption as the peptide-type Hyp hardly takes place. Accordingly, it is necessary to orally ingest collagen peptides for the absorption of the peptide-type Hyp; however, only part of it is absorbed as the peptide-type Hyp. Thus, there has been a problem in that a large amount of collagen peptides must be consumed to obtain the satisfactory effect.

Concerning this problem, it is reported in Patent Literature 1 that the amount of the peptide-type Hyp transferred into the blood increases significantly by the oral ingestion of the collagen peptide that has been digested to a molecular weight of 500 or higher and 2000 or lower with the use of mainly papain. In the collagen peptide, the percentage of hydroxyproline in the second amino acid from the N-terminal is 2-20 mole %, the percentage of glycine at the third amino acid is 20-50 mole %, and the molecular weight is 500-2000. Thus, it is explained that the exposure to various digestive enzymes in the digestive tract becomes adequate, and those in the state of dipeptides/tripeptides increase; as a result, the above-described effect can be achieved.

However, the preparation of the collagen peptides satisfying the above condition is not easy; in addition, there has been a problem in that the time necessary for the digestion of food and drink varies depending upon various factors (fat content of ingested food, individual variation, physical conditions, etc.).

Under these circumstances, a new method that can easily increase the amount of the peptide-type Hyp in blood, which is derived from orally ingested collagen peptide and transferred, into blood, has been desired.

### PRIOR ART DOCUMENTS

### Patent Literatures

### [Patent Literature 1] WO 2010/125910

### [Patent Literature 2] Japanese Unexamined Patent Publication No. 2008-148587

### [Patent Literature 3] Japanese Patent No. 4653052

### Non-Patent Literatures

[Non-patent literature 1] Koyama Yoichi, Hikaku Kagaku (Leather Science), 56: 71-79, 2010 (in Japanese)
[Non-patent literature 2] Iwai K., et al., J. Agric. Food Chem., 53:6531-6536, 2005
[Non-patent literature 3] Shigemura Y., et al., J. Agric. Food Chem., 57:444-449, 2009
[Non-patent literature 4] Ohara H., et al., J. Dermatol., 37:330-338, 2010
[Non-patent literature 5] Nakatani S., et al., Osteoarthritis Cartilage, 17:1620-1627, 2009
[Non-patent literature 6] Kimura Y., et al., Biochem. Biophys. Res. Common., in press, 2014

### SUMMARY OF THE INVENTION

### Problem to be Solved by the Invention

The present invention was made in view of the problems of the above-described conventional art, and an object is to provide a collagen-containing composition for oral ingestion wherein the resulting peptide-type Hyp is effectively transferred into the blood.

### Means for Solving Problem

The present investors have diligently studied to achieve the above-described object. As a result, the present inventors have found that the amount of peptide-type Hyp transferred into the blood increases more drastically when collagen peptide is ingested after mixing with black tea than when it is ingested after mixing with water or other drinks. In addition, the present inventors have found that theaflavins and/or epicatechin contained in black tea forms a complex with collagen peptide and that the collagen peptide in the state of a complex becomes resistant to complete hydrolysis with digestive enzymes, thus leading to the completion of the present invention.

Furthermore, the present inventors have found that when 0.0005-0.09 parts by mass of the theaflavins and/or 0.0001-0.03 parts by mass of epicatechin is mixed with 1 part by mass of water-soluble collagen peptide, a complex having the digestion resistance can be formed effectively.

That is, the present invention comprises the following:

[1] A water-insoluble complex comprising a water-soluble collagen peptide being complexed with theaflavins and/or epicatechin.
[2] The water-insoluble complex according to [1], wherein the water-insoluble complex is formed by mixing 0.0005-0.09 parts by mass of the theaflavins and/or 0.0001-0.03 parts by mass of the epicatechin with 1 part by mass of the water-soluble collagen peptide.
[3] The water-insoluble complex according to [2], wherein the water-insoluble complex is formed by mixing 0.0008-0.01 parts by mass of the theaflavins and/or 0.0002-0.005 parts by mass of the epicatechin with 1 part by mass of the water-soluble collagen peptide.
[4] Food and drink wherein the water-insoluble complex according to any of [1] to [3] is blended.

Drinks that contain collagen peptide and catechins have been reported thus far; however, many of them do not show turbidity and precipitate, and it is considered that no water-insoluble complex is contained. This is because the turbidity and precipitate have been avoided in many drinks because they undermine beauty and texture (for example, Patent Literatures 2 and 3).

### Effect of the Invention

The present invention provides collagen peptide/catechins complexes excellent in the transfer of the peptide-type Hyp into the blood after oral ingestion of the complexes and provides food and drink containing the complexes.

### BRIEF DESCRIPTION OF DRAWINGS

FIG. 1 is a graph that shows the time-dependent change of the concentration of peptide-type Hyp in the blood when the collagen peptide was mixed with various drinks and ingested orally. Each dot represents the average value of the values obtained by five human subjects.

FIG. 2 shows the AUC of the peptide-type Hyp in the blood when the collagen peptide was ingested after mixing with various drinks.

FIG. 3 shows the AUC of free Hyp in the blood when the collagen peptide was ingested after mixing with various drinks.

FIG. 4 shows the AUC of the total Hyp in the blood when the collagen peptide was ingested after mixing with various drinks.

FIG. 5 is a graph that shows the turbidity of mixed solutions wherein 100% black tea extract and a collagen peptide aqueous solution of various concentrations were mixed in the mass ratio of 1:1.

FIG. 6 is a graph that shows the turbidity of mixed solutions when the collagen peptide is mixed with black tea extract of various concentrations so that the final concentration of the collagen peptide will be 20 mg/ml.

FIG. 7 shows the analysis results by reverse-phase chromatography (HPLC charts) for the components of the precipitate that was generated by mixing collagen peptide and black tea drink. A: black tea components before mixing with the collagen peptide, B: black tea components that did not coprecipitate with the collagen peptide, and C: components coprecipitated with the collagen peptide.

FIG. 8 shows the measurement results for generated free Hyp when complexes of collagen peptide and theaflavins or epicatechin were artificially digested in vitro.

FIG. 9 shows the turbidity of the mixed solutions of dry powder of collagen peptide dissolved in artificial gastric juice and dry powder of black tea extract dissolved in artificial gastric juice.

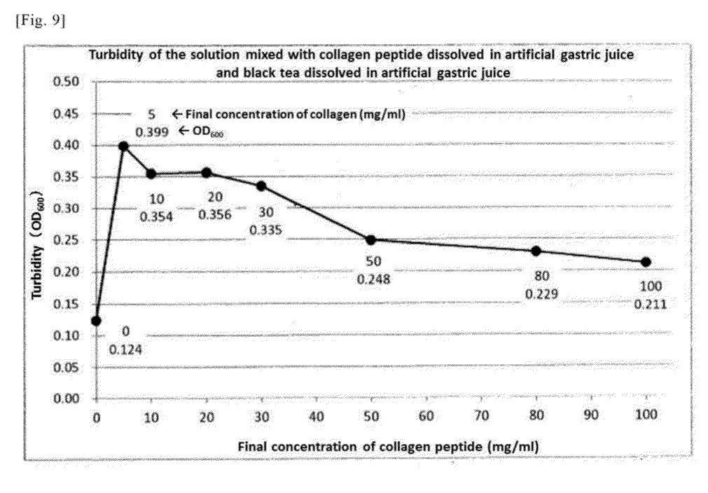

### BEST MODES FOR CARRYING OUT THE INVENTION

Hereinafter, the best modes for carrying out the present invention will be explained. In this patent, “water-soluble” indicates that “a precipitate is not generated when added in water at 25° C.”. On the other hand, “water-insoluble” indicates that “a precipitate is generated when added in water at 25° C.”. In this patent, when the range of a number is expressed with the use of a hyphen, the values in the front and the back of the hyphen are included. For example, the description “the average molecular weight of 3000-5000” means “the average molecular weight of 3000 or higher and 5000 or lower”.

### <Collagen Peptide>

Collagen peptides usable in the present invention are water-soluble peptides produced by hydrolyzing collagen or gelatin. The collagen may be any derived from mammals such as cow and pig; birds such as chicken; or fish such as shark and it is not limited in particular. The hydrolysis can be either the hydrolysis by acid treatment or the hydrolysis with a protease; however, it is preferable to be partial hydrolysis in the case of acid treatment. As the protease that can be suitably used for this purpose, endopeptidase is preferable, and the examples include pepsin, trypsin, chymotrypsin, cathepsin, collagenase, etc.

In the present invention, the collagen peptide whose average molecular weight has become 7000 or less by hydrolysis can be suitably used; the collagen peptide is preferably 1000-6500, more preferably 2100-6000, and most preferably 3000-5000. The average molecular weight is preferably the number average molecular weight and more preferably the number average molecular weight measured by gel permeation chromatography (GPC) method.

### <Theaflavins and Epicatechin>

Theaflavins and epicatechin are tea polyphenols, which are all called catechins. Epicatechin (CAS No.: 490-46-0, molecular weight: 290.3, abbreviated to “EC”) is a catechin originally contained in raw tea leaves (in particular, leaves of Camellia sinensis). On the other hand, theaflavins are catechins generated by oxidative polymerization of tea catechins in the production process of black tea leaves (namely, fermentation process).

Representative theaflavins contained in black tea include theaflavin (CAS No.: 4670-05-7, molecular weight: 564.5, abbreviated to “TF1”), theaflavin-3-O-gallate (CAS No.: 30462-34-1, molecular weight: 716.6, abbreviated to “TF2A”), theaflavin-3′-O-gallate (CAS No.: 28543-07-9, molecular weight: 716.6), and theaflavin-3,3′-O-digallate (CAS No.: 30462-35-2, molecular weight: 868.7, abbreviated to “TF3”). In the present invention, any of these theaflavins can be used.

Catechins originally contained in raw tea leaves (epigallocatechin gallate, epicatechin gallate, epigallocatechin, epicatechin, gallocatechin gallate, catechin gallate, gallocatechin, and catechin) are normally called tea catechins.

<Complex of Collagen Peptide and Theaflavins and/or Epicatechin>

The present invention provides a water-insoluble complex for oral ingestion, which is formed by binding the theaflavins and/or epicatechin with water-soluble collagen peptide, and the resulting peptide-type Hyp is effectively transferred into the blood. “A complex” in this patent indicates an assemblage of two or more materials, in which they are bound through non-covalent bonds. Also, to form a complex is called “complex”.

The complex of collagen peptide and theaflavins and/or epicatechin of the present invention can easily be produced by adding and mixing water-soluble collagen peptide into an aqueous solvent containing theaflavins and/or epicatechin. The aqueous solvent may be pH 3.0-8.5, preferably pH 4.0-8.0, more preferably pH 5.0-7.5, and most preferably pH 6.0-7.0. If the pH is lower than 3.0, the rate of complex formation between collagen peptide and theaflavins and/or epicatechin may decrease. If the pH exceeds 8.5, theaflavins and epicatechin may be denatured. The mixing of the collagen peptide and theaflavins and/or epicatechin can be carried out at about room temperature (specifically at 10-30° C., preferably at 15-25° C., and most preferably at around 20° C.). The aqueous solvent may contain other catechins; furthermore, various components may be contained within the range that the complex formation between collagen peptide and theaflavins and/or epicatechin is not disturbed.

The collagen peptide/theaflavins complex of the present invention can be effectively produced by mixing, in an aqueous solvent, about 0.0005-0.09 parts by mass of theaflavins, preferably about 0.0008-0.01 parts by mass of theaflavins, and more preferably about 0.001-0.007 parts by mass of theaflavins with 1 part by mass of water-soluble collagen peptide. The mass of theaflavins indicated the total amount of one or more theaflavins selected from TF1, TF2A, TF2B, and TF3.

Furthermore, the collagen peptide/epicatechin complex of the present invention can be effectively produced by mixing, in an aqueous solvent, about 0.0001-0.03 parts by mass of epicatechin, preferably about 0.0002-0.005 parts by mass of epicatechin, and more preferably about 0.0005-0.002 parts by mass of epicatechin with 1 part by mass of water-soluble collagen peptide.

Theaflavins and epicatechin can form, without competing, water-insoluble and digestion-resistant complexes by binding to collagen peptide in the presence of each other.

As the aqueous solvent that can be suitably used in the present invention, a black tea extract can be listed. The kinds of black tea leaves usable in the present invention are not limited in particular, and the extract can be produced by a general method.

When a complex of collagen peptide and theaflavins and/or epicatechin is produced with a black tea extract, the collagen peptide may be added and mixed, so that the final concentration of the collagen peptide is about 1-130 mg/ml, preferably about 2.5-100 mg/ml, more preferably about 5-90 mg/ml, and most preferably about 10-80 mg/ml, with the black tea extract produced by the general method (the sum of theaflavins and epicatechin is about 0.05-0.15 mg/ml).

The complex of collagen peptide and theaflavins and/or epicatechin formed in the aqueous solvent can easily be recovered as the precipitate by centrifuging the solution at the centrifugal force of 5,000-10,000×g and preferably at the centrifugal force of about 8,000-10,000×g. Subsequently, dry powder may be formed by a general method (for example, spray drying method or freeze-drying method); a long-term storage is possible at room temperature in the state of dry powder.

### <Peptide-Type Hyp in the Blood>

As described above, when a human orally ingests collagen, gelatin, or collagen peptide, Hyp-containing dipeptides (Pro-Hyp, Leu-Hyp, Hyp-Gly, Ala-Hyp, Glu-Hyp, and Ile-Hyp) and/or Hyp-containing tripeptides (Pro-Hyp-Gly, Ala-Hyp-Gly, and Ser-Hyp-Gly) are detected in the blood (non-patent literature 2). The peptide-type Hyp in the blood of the present invention means these Hyp-containing dipeptides and tripeptides, and the preferable peptide-type Hyp is Pro-Hyp or Hyp-Gly and the most preferable peptide-type Hyp is Pro-Hyp. Hyp (hydroxyproline) in the present invention indicates a compound wherein the 4-position of L-proline is hydroxylated (4-hydroxypyrrolidine-2-carboxylic acid).

“The amount of peptide-type Hyp in the blood (namely, the amount of peptide-type Hyp transferred into the blood)” can be determined by subtracting “the amount of free Hyp in the blood” from “the total amount of Hyp in the blood”.

“The amount of free Hyp in the blood” means “the amount of Hyp present in the blood as a single amino acid”, and it is synonymous with “the amount of Hyp absorbed as an amino acid”. This can be measured with a method known to a person skilled in the art (for example, the method described in non-patent literature 2). For example, a blood plasma sample deproteinized with ethanol (ethanol-soluble fraction) is fractionated by size exclusion chromatography. The obtained amino acid fraction is treated with phenylisothiocyanate (PITC) to convert each amino acid to a phenylthiocarbamyl derivative (PTC-Hyp), and then the amount of PTC-Hyp may be quantified by reverse-phase chromatography.

“The total amount of Hyp in the blood” means “the total amount of free Hyp and Hyp-containing peptides detected in the blood”, and it is synonymous with “the amount of Hyp absorbed into the blood as an amino acid or a peptide”. This can be measured with a method known to a person skilled in the art (for example, Sato K., et al., J. Agric. Food Chem., 40:806-810, 1992). For example, it may be quantified as the amount of PTC-Hyp in the analysis using a reverse-phase column chromatography, in which the blood plasma sample is deproteinized with ethanol, hydrolyzed with hydrochloric acid, treated with PITC, and then subjected to the chromatography.

<Food and Drink Wherein a Complex of Collagen Peptide and Theaflavins and/or Epicatechin is Blended>

The present invention provides various food and drink wherein a complex of collagen peptide and theaflavins and/or epicatechin is blended. The complex is excellent in transferring the peptide-type Hyp into the blood after oral ingestion (about two times of the rate when the same amount of non-complexed collagen peptide is orally ingested). By the oral ingestion of food and drink containing the complex, the effects such as the alleviation of joint pain, an increase in bone density, a reduction of skin damage by ultraviolet irradiation (decrease in the stratum corneum moisture content, epidermal hyperplasia, reduction of dermal collagen, etc.) are expected to be obtained with a less amount of collagen peptide intake that) in the past (about a half amount).

In the present invention, the embodiment of the complex of collagen peptide and theaflavins and/or epicatechin includes the case in that the complex is food or drink itself and the case in that the complex is a raw material or an intermediate product in the production of food and drink.

The form of food and drink, wherein the complex of collagen peptide and theaflavins and/or epicatechin of the present invention is blended, may be either solid, semi-solid (jelly/gel-like), or liquid. Examples of the drinks include tea-based drinks, coffee drinks, carbonated drinks, fruit drinks, mineral water, soy milk drinks, vegetable drinks, spoils drink, milk drinks, etc. The preferable examples of food include confectionery, dairy products, noodles, marine/animal processed food, oil/fat processed products, etc.

In the present invention, the blending quantity of the complex of collagen peptide and theaflavins and/or epicatechin in the food and drink may be the amount that can achieve the physiological effect and pharmacological effect. When we consider the amount of normal intake of the target food and drink, it is desirable that the amount of intake per day for an adult is normally 100-10,000 mg, preferably 500-6,000 mg, and more preferably 1,000-3,000 mg. For example, it can be 1-90 mass % in the case of solid food and about 0.1-20 mass % in the case of liquid food such as drink.

In the food and drink of the present invention, components normally used in food and drink can be suitably blended within the range that the binding of collagen peptide and theaflavins and/or epicatechin is not disturbed.

### EXAMPLES

Hereinafter, the present invention will be explained in further detail with reference to examples. However, the scope of the present invention is not limited by these examples. Initially, the representative methods used in the below Test Examples 1 to 5 will be explained. In the below-described test examples, the centrifugal operation for all the samples was carried out at room temperature (about 20° C.).

### Method 1

### Preparation Method of a Collagen Peptide Aqueous Solution

Collagen peptide (dry powder of the pig skin-derived collagen hydrolysate, the average molecular weight: 3000-5000, manufactured by Nippi, Inc.) was dissolved in water so that the specified concentration is achieved.

### Method 2

### Preparation Method of a Black Tea or Green Tea Extract

To 180 ml of hot water at about 90° C., 2.1 g of commercially available black tea leaves (black tea leaves A: Lipton Yellow Label tea bags (manufactured by Unilever Japan K.K.), black tea leaves B: Nittoh black tea Day & Day (manufactured by Mitsui Norin Co., Ltd.)), or green tea leaves (Oi Ocha (manufactured by Ito En, Ltd.)) was added, the extraction was carried out with stirring for 3 minutes, and then the supernatant was obtained by filtration. In the examples of the present patent application, “supernatant” obtained by the above-described method is used as “solvent”, “drink”, or “extract” to be mixed with collagen peptide. The supernatant was cooled to room temperature (25° C.) and then mixed with a collagen peptide aqueous solution of the designated concentration.

In this patent, “black tea” and “green tea” means “black tea drink” and “green tea drink”, respectively. Tea leaves, which are the raw materials for these drinks are distinguished by calling them “black tea leaves” and “green tea leaves”.

### Method 3

### Measurement Method for Peptide-Type Hyp, Free Hyp, and the Total Hyp in the Blood

Blood plasma sample was deproteinized with ethanol (ethanol-soluble fraction) and subjected to a size exclusion chromatography (column: Superdex Peptide 10/300GL (manufactured by GE health Care) to obtain the fraction containing peptides and amino acids. The peptide and amino acid containing fraction was hydrolyzed with hydrochloric acid (under reduced pressure, 150° C., 1 hour), treated with PITC, and then subjected to a reverse-phase chromatography (column: LiChroCART 250-4 (manufactured by Merck)) to determine the amount of PTC-Hyp (=the total amount of Hyp in the blood). The peptide and amino acid containing fraction was also subjected to the PITC treatment followed by the reverse-phase chromatography without hydrochloric acid hydrolysis, and the amount of PTC-Hyp (=the amount of free Hyp in the blood) was determined. The value obtained by subtracting “the amount of free Hyp in the blood” from “the total amount of Hyp in the blood” was regarded as “the amount of peptide-type Hyp in the blood”.

### Method 4

### Measurement Method of Free Hyp and the Total Hyp in an Aqueous Solution

A supernatant was obtained by adding the triple volume (v/v) of ethanol to the aqueous solution and centrifuging (10,000×g, 15 min). The supernatant was hydrolyzed with hydrochloric acid (under reduced pressure, 150° C., and 1 hour). After the PITC treatment, the amount of PTC-Hyp (=the total amount of Hyp in the solution) was determined by carrying out reverse-phase chromatography (column: LiChroCART 250-4 (manufactured by Merck)). The amount of PTC-Hyp (=the amount of free Hyp) was determined by carrying out the PITC treatment/reverse-phase chromatography for the supernatant without hydrochloric acid hydrolysis.

### Test Example 1

### Investigation of Solvent to Dissolve Collagen Peptide

The present inventors have carefully investigated the ingestion method of collagen peptide to increase the amount of absorbed peptide-type Hyp when a human orally ingests the collagen peptide. As a result, present inventors have found that the amount of peptide-type Hyp in the blood greatly varies depending upon the solvent to which collagen peptide is added.

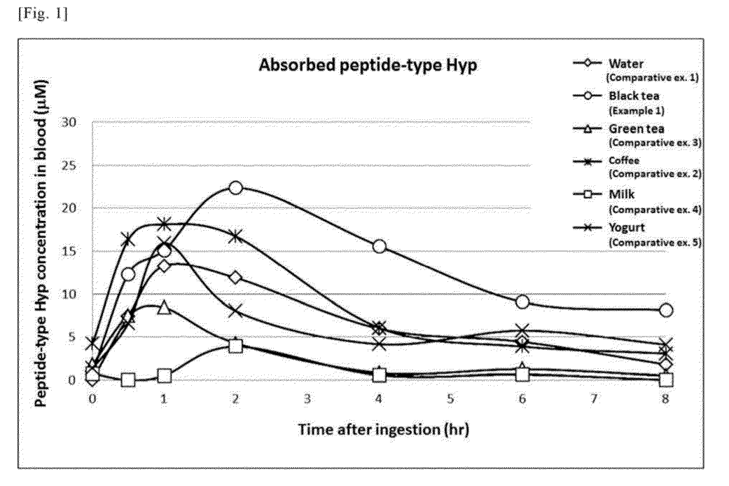

Collagen peptide was added and mixed, so that the final concentration will be 25 mg/ml, to six kinds of solvent (water, milk, black tea, green tea, coffee, or yogurt drink) (200 ml each, 220 ml in the case of yogurt drink to keep the amount of protein in line with that of milk), and five human subjects were asked to ingest them orally. Blood was collected from the human subjects before ingestion (0 hour) and after ingestion (after 0.5, 1, 2, 4, 6, and 8 hours). The total amount of Hyp in the blood, the amount office Hyp in the blood, and the amount of peptide-type Hyp in the blood were measured and calculated according to Method 3. The amount of peptide-type Hyp in the blood is shown in FIG. 1.

As shown in FIG. 1, when collagen peptide was ingested after added to water (Comparative Example 1), the amount of peptide-type Hyp in the blood increased over about 1 hour alter the ingestion, and then it suddenly decreased. On the other hand, when collagen peptide was ingested after added to black tea (Example 1) or coffee (Comparative Example 2), the increase in the amount of the peptide-type Hyp in the blood was faster and the subsequent decrease was more gradual than when ingested after added to water. Furthermore, when collagen peptide was added to green tea (Comparative Example 3), milk. (Comparative Example 4), or yogurt drink (Comparative Example 5), no such effect was observed. Rather, the increase of the peptide-type Hyp in the blood became very slow when collagen peptide was added to milk.

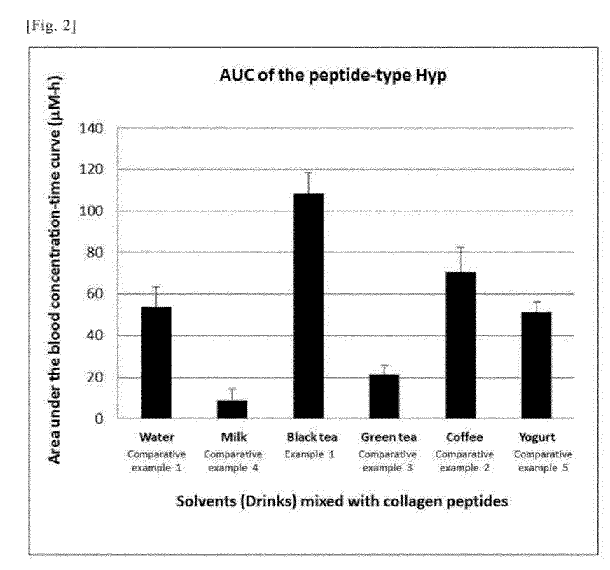

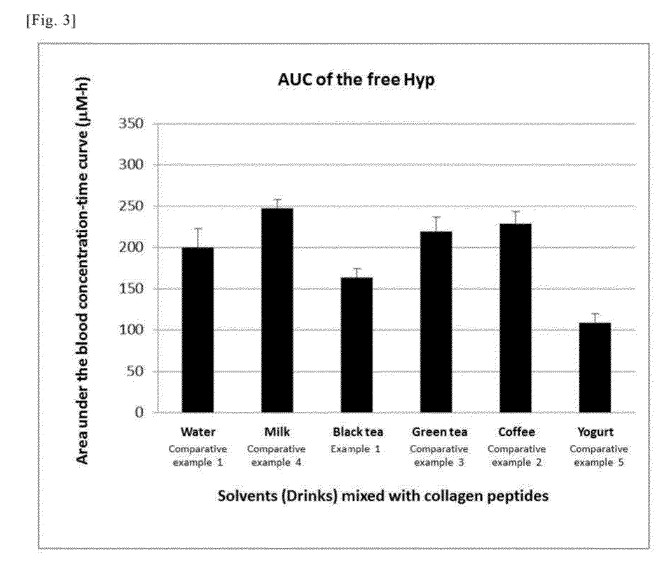

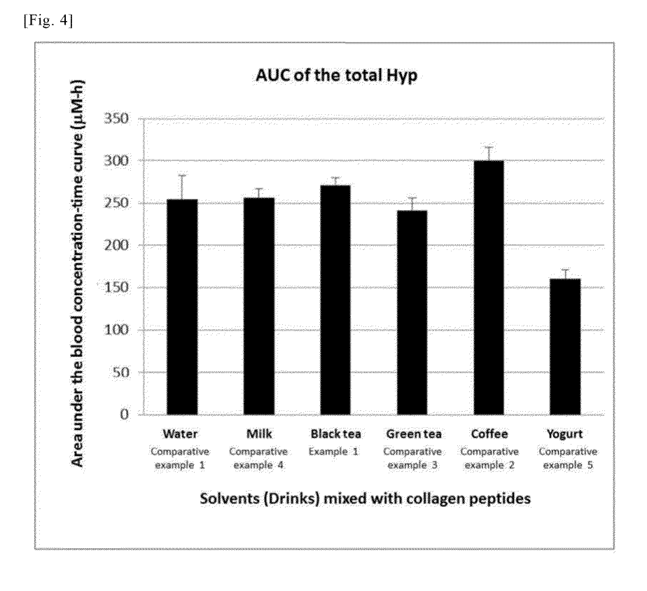

From the graph in FIG. 1, the AUC (area under the blood concentration-time curve) of the peptide-type Hyp transferred into the blood was calculated and the results are shown in FIG. 2. Similarly, the ADC was calculated for free Hyp in the blood and for the total Hyp in the blood, and the results are shown in FIG. 3 and FIG. 4, respectively. From FIG. 2, it was clarified that when collagen peptide was added and mixed with black tea and orally ingested, the amount of peptide-type Hyp transferred into the blood increased about two times compared with the amount when collagen peptide was added and mixed with water and orally ingested.

The AUC value of the total Hyp in the blood (FIG. 4) represents “the total amount of collagen peptide-derived amino acids, dipeptides, and tripeptides” absorbed in the small intestine. There was no significant difference between the AUC value of the total Hyp in the blood when black tea, coffee, milk, or green tea was used as the solvent and the AUC value when water was used as the solvent. Therefore, it is considered that no drastic difference is present in “the total amount of collagen peptide-derived amino acids, dipeptides, and tripeptides” absorbed in the small intestine when collagen peptide is added to these solvents and ingested. Nevertheless, when black tea was used as the solvent, the amount of free Hyp in the blood decreased significantly (FIG. 3), and the amount of peptide-type Hyp in the blood increased significantly (FIG. 2). Accordingly, these results suggests that, when collagen peptide is orally ingested after mixing with black tea, a percentage of dipeptides or tripeptides (without being hydrolyzed to amino acids) in “the total amount of collagen peptide-derived amino acids, dipeptides, and tripeptides” in the small intestine increase compared with the percentage when collagen peptide is orally ingested after mixing with water or other drinks.

### Test Example 2

### Change of Solvent Turbidity by the Addition of Collagen Peptide

The reason why collagen peptide becomes resistant to digestion when it was mixed with black tea was investigated. In Test Example 1, when collagen peptide was mixed wish water, green tea, or coffee, pronounced turbidity was not observed. However, marked turbidity was observed when collagen peptide was mixed with black tea (unknown for milk and yogurt drinks). Therefore, this turbidity was investigated in detail.

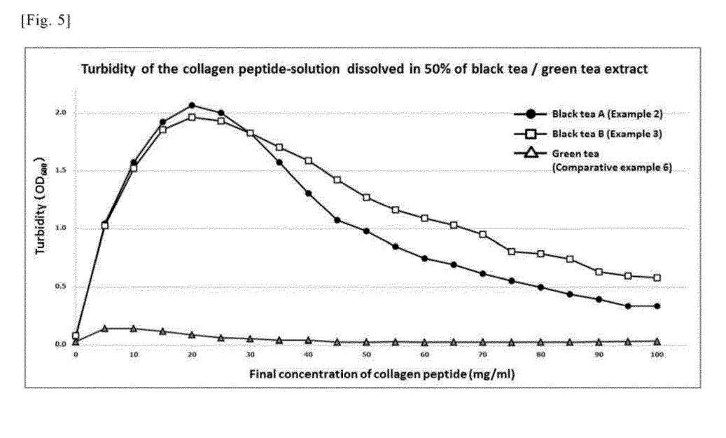

The extract from black tea leaves A or B or the extract from green tea leaves was mixed with the collagen peptide aqueous solution of various concentrations (0-200 mg/ml) at the mass ratio of 1:1, and the absorbance at 600 nm (namely, turbidity) was measured. The results are shown in FIG. 5.

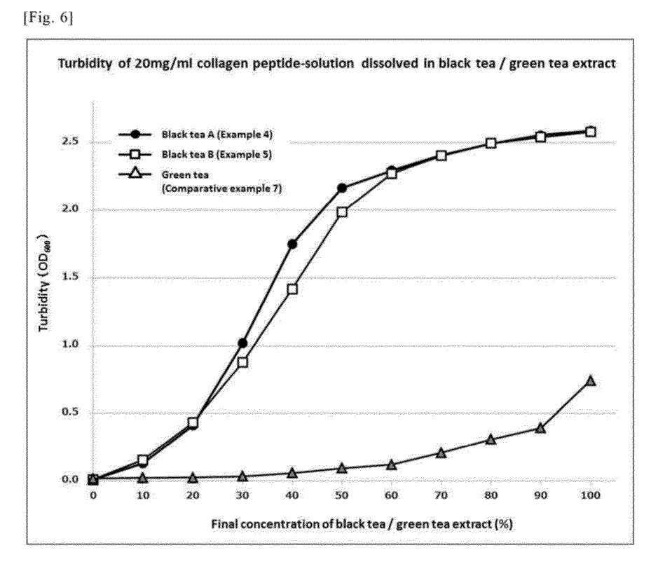

Interestingly, when the collagen peptide was mixed with black tea, the turbidity drastically rose with an increase in the amount of the peptide; however, the maximum was reached at 20 mg/ml (final concentration). When the amount of the peptide was further increased, the turbidity turned to decline (FIG. 5, Examples 2 and 3). That is, the increase in the turbidity may not be attributable to a non-specific aggregation accompanied with an increase of polymeric material, but may be attributable to a relatively specific interaction between the collagen peptide and a specific component in the black tea. Then, the final concentration of black tea was varied from 100% to 0% while the amount of collagen peptide was fixed at 20 mg/ml (final concentration); as a result, sigmoidal curves were obtained as shown in FIG. 6 (Examples 4 and 5). The test tor the final concentration of 100% was carried out by directly adding dry collagen powder to 100% black tea extract.

These results suggest that the collagen peptide can form a water-insoluble complex by interacting with a specific component in black tea.

When the collagen peptide was mixed with green tea, a substantial increase in the turbidity was not observed in the wide range of the final peptide concentration, namely 0-100 mg/ml (Comparative Example 6 in FIG. 5 and Comparative Example 7 in FIG. 6).

### Test Example 3

### Identification of Black Tea Components that Form Complexes with Collagen Peptide

Subsequently, the identification of black tea components that binds to the collagen peptide was carried out.

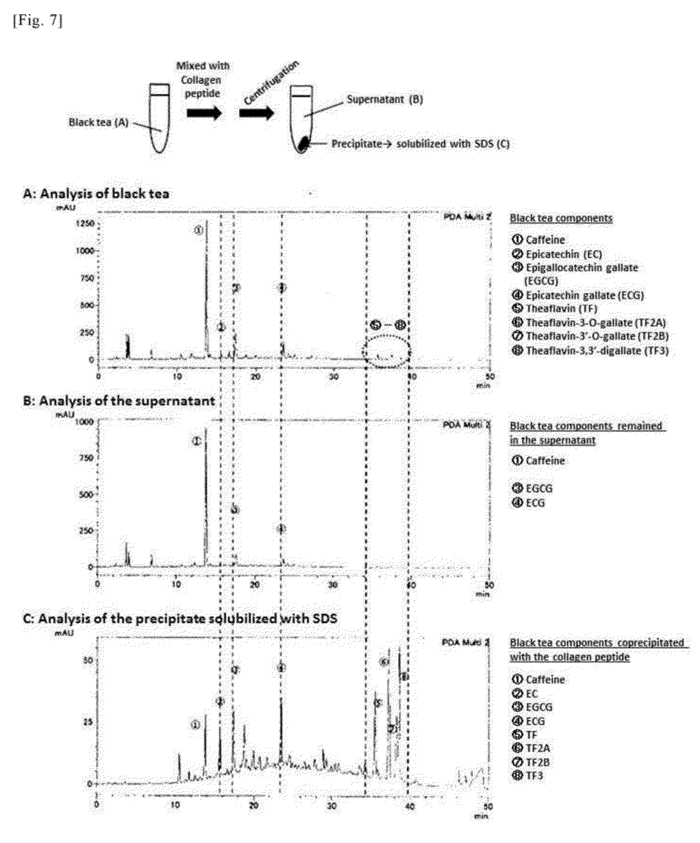

To black tea A (200 ml), collagen peptide (5 g) was added and mixed; the supernatant and precipitate were separated by centrifuging (10,000×g, for 10 minutes) (Example 6). To determine the components, of the black tea A before the addition of the collagen peptide (FIG. 7A), the supernatant after centrifuging (FIG. 7B), and the precipitate solubilized with SDS (FIG. 7C), were analyzed by reverse-phase chromatography (column: Cosmosil 5C18-AR-II (manufactured by Nacalai Tesque, Inc.), mobile phase: 0.1% formic acid-containing acetonitrile, detection: 215 nm, 280 nm), respectively. HPLC charts of reverse-phase chromatography for the respective solutions are shown in FIGS. 7A-7C. FIG. 7A is for the components contained in the black tea, FIG. 7B is for the components that did not coprecipitate with the collagen peptide among the black tea components FIG. 7C is for the components coprecipitated with the collagen peptide among the black tea components.

Furthermore, the contents of the representative polyphenols were analyzed for black tea A, black tea B, and green tea (collagen peptide was not added), and the results are shown in Table 1.

TABLE 1

| | Content per 1 ml of drink, mg (% of solid material) | | |
|---|---|---|---|
| Components | Black tea A | Black tea B | Green tea |
| Solid materials | 4.100 (100.0%) | 4.600 (100.0%) | 4.100 (100.0%) |
| (mg) | | | |
| Tea catechins, | 0.122 (2.98%) | 0.282 (6.14%) | 0.575 (14.02%) |
| total | | | |
| Epicatechin (EC) | 0.010 (0.23%) | 0.028 (0.62%) | 0.052 (1.27%) |
| Theaflavins, | 0.065 (1.59%) | 0.064 (1.40%) | 0.0 (0.0%) |
| total | | | |
| Theaflavin (TF1) | 0.021 (0.52%) | 0.028 (0.62%) | 0.0 (0.0%) |
| Theaflavin | 0.015 (0.38%) | 0.014 (0.31%) | 0.0 (0.0%) |
| monogallate | | | |
| (TF2A + TF2B) | | | |
| Theaflavin | 0.029 (0.70%) | 0.022 (0.47%) | 0.0 (0.0%) |
| digallate (TF3) | | | |

FIG. 7A and Table 1 show that, in the black tea, tea catechins such as [1] caffeine, [2] epicatechin, [3] epigallocatechin gallate, and [4] epicatechin gallate are present in a large amount, but [5] theaflavins such as theaflavin (TF1), [6] theaflavin-3-O-gallate (TF2A), [7] theaflavin-3′-O-gallate (TF2B), and [8] theaflavin-3,3′-O-digallate (TF3) are present in considerably less amount than caffeine and tea catechins. FIG. 7B indicates that components [1], [3], and [4] remained mostly in the supernatant, from which the precipitate had been removed, while [2] epicatechin and theaflavins ([5]-[8]) disappeared from the supernatant. Furthermore, FIG. 7C indicates that almost all the disappeared components, [2] and [5]-[8], coprecipitated with collagen.

Parts of the above-described [1], [3], and [4] were also shown to coprecipitate with the collagen (FIG. 7C); however, all of these are components normally contained more in green tea than in black tea. Because the turbidity did not increase when the collagen peptide was mixed with green tea (FIGS. 5 and 6), it is considered that these components (caffeine, epigallocatechin gallate, and epicatechin gallate) cannot form a water-insoluble complex enough even when they bind to the collagen peptide.

Thus, it was clarified that theaflavins (TF1, TF2A, TF2B, and TF3) and/or epicatechin contained in black tea forms a water-insoluble complex by binding to collagen peptide.

As shown in FIG. 5, when the final concentration of the collagen peptide in the 50 mass % black tea aqueous solution was 5-65 mg/ml and especially when it was 10-40 mg/ml, sufficient water-insoluble complexes were formed and the turbidity increased markedly. From Table 1, about 0.032 mg-0.033 mg of theaflavins (average: 0.032 mg) is contained in 1 ml of 50 mass % black tea aqueous solution. Thus, it was clarified that a water-insoluble collagen peptide/theaflavin complex can be effectively formed when about 150-2000 parts by mass of collagen peptide is mixed with 3 part by mass of theaflavins and preferably when about 300-1200 parts by mass of collagen peptide is mixed with 1 part by mass of theaflavins. The mixing ratio of theaflavins is about 0.0005-0.0070 parts by mass and preferably 0.0008-0.0033 parts by mass with respect to 1 part by mass of water-soluble collagen peptide.

Similarly, about 0.005 mg-0.014 mg (average: 0.010 mg) of epicatechin is contained in 1 ml of 50 mass % black tea aqueous solution. Thus, it was shown that a water-insoluble collagen peptide/epicatechin complex can be effectively formed when about 500-6500 parts by mass of collagen peptide is mixed with 1 part by mass of epicatechin and preferably when about 1000-4000 parts by mass of collagen peptide is mixed with 1 part by mass of epicatechin. The mixing ratio of epicatechin is about 0.0001-0.0020 by mass and preferably 0.0002-0.0010 parts by mass with respect to 1 part by mass of water-soluble collagen peptide.

### Test Example 4

### Complex Formability of Theaflavins and Epicatechin with Collagen Peptide

Subsequently, it was investigated whether the respective theaflavins or epicatechin can independently form a water-insoluble complex with collagen peptide.

The respective aqueous solutions of theaflavins and epicatechin (1 mg/ml) and collagen peptide aqueous solution (200 mg/ml) were prepared, and they were raised at room temperature so that the below-described contents are attained. In the test examples of the present patent application, the following reagents were used.

### <Theaflavins and Epicatechin>

TF1: theaflavin, TF2A: theaflavin-3-gallate, TF2B: theaflavin-3′-gallate, TF3: theaflavin-3,3′-digallate (all of them are manufactured by Wako Pure Chemical Industries, Ltd.) epicatechin: (−)-Epicatechin (manufactured by Sigma-Aldrich Co. LLC.)

### <In 1 ml of Reaction Solution>

| | |
|---|---|
| TF1 (about 0.113 mg), TF2A (about 0.143 mg), | 0.2 μmol |
| TF2B (about 0.143 mg), TF3 (about 0.174 mg), | |
| or epicatechin (about 0.058 mg) | |
| Collagen peptide | 2 mg or 10 mg |
| Purified water | balance |

After mixing, the supernatant and precipitate were separated by centrifuging (8,000×g, 15 mm). According to Method 4, the total amount of Hyp was determined for the supernatant (=“the total amount of Hyp in the supernatant”). “The total amount of Hyp in the complex” was calculated by subtracting “the total amount of Hyp in the supernatant” from the amount of collagen peptide used in the above reaction (2 mg or 10 mg). The respective total amounts of Hyp were expressed in percentage (%), and the results are shown in Table 2.

TABLE 2

| | | Collagen peptide 2 mg | | Collagen peptide 10 mg | |
|---|---|---|---|---|---|
| | | Total Hyp | Total Hyp | Total Hyp | Total Hyp |
| | | in the su- | in the | in the su- | in the |
| | | pernatant (%) | complex (%) | pernatant (%) | complex (%) |
| Epicatechin (Example 7) | | 6.9 | 93.1 | 11.6 | 88.4 |
| Theaflavins | TF1 (Example 8) | 13.5 | 86.5 | 16.2 | 83.8 |
| | TF2A (Example 9) | 13.0 | 87.0 | 13.4 | 86.6 |
| | TF2B (Example 10) | 2.3 | 97.7 | 9.5 | 90.5 |
| | TF3 (Example 11) | 2.0 | 98.0 | 2.2 | 97.8 |

From Table 2, it was confirmed that any of theaflavins and epicatechin alone can bind to collagen peptide and form a water-insoluble complex effectively (Examples 7 to 11). In particular, TF2B (Example 10) and TF3 (Example 11) are highly capable of forming a complex with collagen peptide; it was shown that 90% or higher of collagen peptide forms a water-insoluble complex in the presence of these theaflavins.

Furthermore, it was confirmed from the above results that a water-insoluble complex consisting of a catechin and collagen peptide can be formed by adding and mixing only a minute amount of collagen peptide to the above-described respective catechins. The mass ratio of the respective catechins and collagen peptide used in the above reaction is about 0.0113 mg or 0.0565 mg of TF1, about 0.0143 mg or 0.0716 mg of TF2A/TF2B, about 0.0174 mg or 0.0870 mg of TF3, and about 0.0058 mg or 0.0290 mg of epicatechin with respect to 1 mg of collagen peptide. Accordingly, it was confirmed that a water-insoluble complex can be obtained by mixing up to about 0.090 part by mass of each theaflavin or up to about 0.030 part by mass of epicatechin with respect to 1 part by mass of collagen peptide.

From the results of the above-described Test Example 4 (Table 2) and Test Example 2 (FIG. 5), it was clarified that a water-insoluble collagen peptide/theaflavin complex can be effectively formed by mixing about 0.0005-0.09 parts by mass of theaflavins (the total amount of theaflavins), preferably about 0.0008-0.01 parts by mass thereof, and more preferably about 0.001-0.007 parts by mass thereof with respect to 1 part by mass of water-soluble collagen peptide.

Similarly, it was shown that a water-insoluble collagen peptide/epicatechin complex can be effectively formed by mixing about 0.0001-0.03 parts by mass of epicatechin, preferably about 0.0002-0.005 parts by mass thereof, and more preferably about 0.0005-0.002 parts by mass thereof with respect to 1 part by mass of water-soluble collagen peptide.

### Test Example 5

### Digestion Resistance of the Complex of Collagen Peptide and Theaflavins and/or Epicatechin

From the results of Test Examples 1-4, a possibility that the collagen peptide becomes resistant to digestion by the formation of a complex with theaflavins and/or epicatechin is suggested. Therefore, the digestion resistance of the complex was investigated.

With the use of the same method as Test Example 4, 1 mg each of theaflavins (TF1, TF2A, TF2B, or TF3) or epicatechin and 2.5 mg of the collagen peptide were mixed and a complex was formed. A solution containing only the collagen peptide was used as the negative control. To these solutions, pancreatin (panecreatin (manufactured by Wako Pure Chemical industries, Ltd.), 5.2-9.2 U), leucine aminopeptidase (Leucine Aminopeptidase, microsomal from porcine kidney (manufactured by Sigma-Aldrich Co. LLC), 2.8 U), and carboxypeptidase (Carboxypeptidase A from bovine pancreas (manufactured by Sigma-Aldrich Co. LLC), 17.8 U) were added, and the digestion of the collagen peptide was carried out by incubating in 1 M Tris buffer (pH 8.0) at 37° C. for 48 hours (volume of the reaction system: 300 μl). The reaction system is an artificial digestion system simulating a digestion of proteins occurred in the duodenum and the small intestine. Considering that food and drink normally pass through the duodenum and the small intestine in about 1-6 hours, the artificial digestion is thought to be an intense digestion under very severe conditions.

After the incubation, the reaction solution was centrifuged (10,000×g, 15 min), the complex was removed as a precipitate, and the supernatant was recovered. The amount of free Hyp contained in the supernatant was determined according to Method 4. The results are shown in Table 3.

TABLE 3

| | Free Hyp in the |
|---|---|
| Complex | supernatant, nmol/ml (%) |
| Collagen peptide alone (control) | 36.026933 (100.0%) |
| (Comparative example 8) | |
| Complex of collagen peptide and TF1 | 25.544267 (70.9%) |
| (Example 12) | |
| Complex of collagen peptide and TF2A | 20.188267 (56.0%) |
| (Example 13) | |
| Complex of collagen peptide and TF2B | 14.973600 (41.6%) |
| (Example 14) | |
| Complex of collagen peptide and TF3 | 18.677600 (51.8%) |
| (Example 15) | |
| Complex of collagen peptide and | 15.958933 (44.3%) |
| epicatechin (Example 16) | |

* Total Hyp in the reaction solution before the digestion was 2.8292583 μmol/ml.

As seen in Table 3, when the collagen peptide was artificially digested after the complex formation with theaflavins or epicatechin, the amount of free Hyp recovered in the supernatant (namely, the amount of collagen peptide hydrolyzed to amino acids) decreased to about 44.3-70.9% compared with when the collagen peptide alone was artificially digested. As described above, the artificial digestion reaction is a very severe reaction; therefore, the complex of collagen peptide and theaflavins or epicatechin was shown to have strong resistance to digestion (in particular, in the duodenum and the small intestine).

Accordingly, it was shown that collagen peptide becomes resistant to digestion with digestive enzymes in the duodenum and pancreatic juice by forming a water-insoluble complex with theaflavins and/or epicatechin, thus resulting in a decrease of complete digestion of the collagen peptide into amino acids.

Furthermore, the present inventors found that the complex should be formed prior to oral ingestion, because collagen peptide cannot effectively form the complex with theaflavins in the artificial gastric juice.

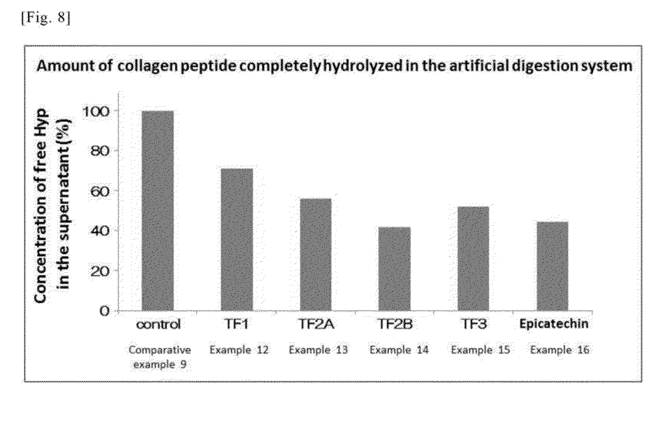

Solutions were prepared by dissolving the freeze-dried powder of black tea A extract and the dry powder of collagen peptide in artificial gastric juice (0.2% NaCl aqueous solution, pH 1.2), respectively. They were mixed so that the final concentration as the black tea extract is 50 mass % and the final concentration of the collagen peptide is 0-100 mg/ml, and then the turbidity (absorbance at 600 nm) of the mixed solution was measured. That is, it was a test wherein the solvent was changed from “water” to “artificial gastric juice” in the analysis of Test Example 2 of the present patent application (FIG. 5). The results are shown in FIG. 8; the increase in turbidity was very small unlike FIG. 5 (about one fifth of that of FIG. 5 even when the maximum values are compared). Thus, it was shown that the collagen peptide and the black tea components cannot effectively form complexes in the artificial gastric juice.

From the results of Test Examples 1 to 5, it was shown that a water-soluble collagen peptide forms a water-insoluble complex with theaflavins and/or epicatechin and that the amount of peptide-type Hyp transferred into blood markedly increases when the complex is orally ingested.

In the following, the examples of food and drink, wherein the complex of collagen peptide and theaflavins and/or epicatechin of the present invention is blended, are listed. However, the present invention is not limited to these examples. The following blending quantities are all expressed in mass %.

In the following, the examples of food and drink, wherein the complex of collagen peptide and theaflavins and/or epicatechin of the present invention is blended, are listed. However, the present invention is not limited to these examples. The following blending quantities are all expressed in mass %.

### Example 17

### Vegetable Juice

### <Formulation>

| | |
|---|---|
| Component | Blending quantity |

| | | |
|---|---|---|
| (1) | Collagen peptide (IXOS HDL-50, | 0.4 |
| | average molecular weight: 5000, | |
| | manufactured by Nitta Gelatin Inc.) | |
| (2) | Black tea extract | 10.0 |
| (3) | Vegetable squeeze | 70.0 |
| (4) | Five-times concentrated apple juice | 5.0 |
| (5) | Three-times concentrated lemon juice | 2.0 |
| (6) | Sodium ascorbate | 0.05 |
| (7) | Purified water | balance |

| | |
|---|---|
| Sum | 100.0 |

### <Preparation Method>

Component (1) was dissolved in 10 ml of (7), and then it was mixed with (2). To the mixed solution, the residual amount of (7) and (3)-(6) was mixed to obtain vegetable juice.

### Example 18

### Tablet

### <Formulation>

| Component | | Blending quantity |
|---|---|---|
| (1) | Collagen peptide/theaflavin complex | 10.0 |
| | Dry powder of precipitate obtained in Example 6 | |
| (2) | Lactose | 75.0 |
| (5) | Sodium ascorbate | 10.0 |
| (6) | Dried corn starch | 3.0 |
| (7) | Talc | 1.8 |
| (8) | Sodium stearate | 0.2 |

| | |
|---|---|
| Sum | 100.0 |

### <Preparation Method>

A mixture wherein components (1) to (8) were uniformly mixed was compressed with a single punch tableting machine, and tablets with a diameter of 5 mm and a mass of 15 mg were obtained.

### INDUSTRIAL APPLICABILITY

The complex of collagen peptide and theaflavins and/or epicatechin of the present invention can be easily produced and stably stored for a long term; therefore, it is very valuable as a raw material for food and drink. By blending the complex, food and drink having a pharmacological effect of collagen and excellent in the absorption of “peptide-type Hyp” into blood can be easily produced.

What is claimed is:

1. A water-insoluble complex comprising a water-soluble collagen peptide being completed with theaflavins and/or epicatechin.

2. The water-insoluble complex according to claim 1, wherein the complex is formed by mixing 0.0005-0.09 parts by mass of theaflavins and/or 0.0001-0.03 parts by mass of epicatechin with 1 part by mass of the water-soluble collagen peptide.

3. The water-insoluble complex according to claim 2, wherein the complex is formed by mixing 0.0008-0.01 parts by mass of theaflavins and/or 0.0002-0.005 parts by mass of epicatechin with 1 part by mass of the water-soluble collagen peptide

4. Food and drink comprising the water-insoluble complex according to claim 1.

5. Food and drink comprising the water-insoluble complex according to claim 2.

6. Food and drink comprising the water-insoluble complex according to claim 3.
